(19) United States
(12) Patent Application Publication
(10) Pub. No.: US 2005/0256463 A1
(43) Pub. Date: Nov. 17, 2005

(54) Cosmetic apparatus

(76) Inventor: Masatoshi Masuda, Kyoto-city (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/828,899

(22) Filed: Apr. 21, 2004

(51) Int. Cl. A61H 1/00; A61H 1/02; A61H 5/00; A61N 1/30; A61B 17/20; A61M 35/00; A61K 9/22

(52) U.S. Cl. 604/290; 604/20; 604/22; 604/890.1

(57) ABSTRACT

A cosmetic apparatus comprises a conductive pad; at least one ultrasonic transducer placed on the pad, which is configured to be connected to an ultrasonic wave signal generator to transmit ultrasonic waves to a skin surface through the conductive pad; and a low- and/or moderate-frequency current terminal connected to the conductive pad, which is configured to be connected to a low- and/or moderate-frequency current generator to transmit a low- and/or moderate-frequency current to the skin surface through the conductive pad.

### BACKGROUND OF THE INVENTION

The present invention generally relates to a cosmetic apparatus, which brings about cosmetic effect and slimming effect by bringing a pad, to which ultrasonic vibration and low-frequency current or moderate-frequency current are fed, into contact with the skin.

As an ultrasonic-vibration-application-type cosmetic apparatus, which brings about cosmetic effect by applying ultrasonic vibration to a skin, an apparatus possessing an ultrasonic transducer feeding ultrasonic vibration to the skin is known. A low-frequency-application-type cosmetic apparatus, which brings about slimming effect by stimulating the muscle layer by applying low-frequency current to the skin has been proposed.

### SUMMARY OF THE INVENTION

Conventional low-frequency-application-type cosmetic apparatuses have the following problem: Because they are constructed to apply low-frequency current to the skin by a pad attached to the skin, a position of the pad is always fixed; hence, a stimulus is given only to the same area of the muscle layer; it is impossible to bring about slimming effect to a broad area in the entire body.

The present invention has been achieved to solve the above-mentioned problem. An object of the present invention is to provide a cosmetic apparatus which can achieve slimming effect in a broad area in the entire body while bringing about cosmetic effect by feeding a low-frequency current to a pad which applies ultrasonic vibration to the skin while sliding against the skin, or a number of pads which apply ultrasonic vibration to the skin successively.

Additionally, only by applying low-frequency current to the skin, it is impossible to give a stimulus down to the muscle layer, which is a deep layer. Another object of the present invention is to provide a cosmetic apparatus, which can achieve slimming effect more effectively by giving a stimulus down to the muscle layer.

In an embodiment, the present invention provides a cosmetic apparatus comprising: (i) at least one conductive pad; (ii) multiple ultrasonic transducers placed on the at least one conductive pad, which is configured to be connected to an ultrasonic wave signal generator to transmit ultrasonic waves to a skin surface through the conductive pad, wherein each ultrasonic transducer is provided with at least one conductive pad; and (iii) a low- and/or moderate-frequency current terminal connected to the at least one conductive pad, which is configured to be connected to a low- and/or moderate-frequency current generator to transmit a low- and/or moderate-frequency current to the skin surface through the at least one conductive pad. By a combination of ultrasonic waves and a low- and/or moderate-frequency current through a common pad, and by using multiple ultrasonic transducers, cosmetic effects can effectively and easily be obtained including body metabolism stimulatory effect by a massage given and deep thermal action by thermal energy effects, which are brought about by propagating ultrasonic vibration oscillated by the ultrasonic transducer to the skin via the pad, as well as slimming effect by stimulating the muscle layer by applying low- and/or moderate-frequency current to the skin via the pad.

The above embodiments further include the following embodiments, although the present invention should not be limited to these embodiments, wherein elements used in one embodiment can interchangeably be used in another embodiment:

The at least one conductive pad may be one conductive pad, and the multiple ultrasonic transducers may be placed on the one conductive pad. The multiple ultrasonic transducers may be arranged next to each other symmetrically around a center of a surface of the pad facing the skin surface. The surface of the pad may have a circular shape.

The cosmetic apparatus may comprise multiple conductive pads each provided with multiple ultrasonic transducers and a low- and/or moderate-frequency current terminal. The low- and/or moderate-frequency current terminal can be provided in the ultrasonic transducer, wherein the ultrasonic transducer functions also as the low- and/or moderate-frequency current terminal, to which a low- and/or moderate frequency current can be supplied through a cord connecting the ultrasonic transducer which is connected to the conductive pad, for example.

The pads may have different shapes or different colors. For example, one is configured to be used for facial treatment, and the other is configured to be used for deeper treatment such as stimulation of muscles.

In the above, the cosmetic apparatus may further comprise a gripper configured to be held by a hand.

In an embodiment, one pad is provided per ultrasonic transducer, and the cosmetic apparatus comprises a plurality of pads and a support on which the plurality of pads are arranged. The support may have a longitudinal shape, and the cosmetic apparatus further comprises a belt onto which the support is attached. In this embodiment, the pads need not slide against the skin surface because the pads can be used in sequential cycles so that it can avoid applying energy to the same spot of the skin for a long period of time.

In the above, the cosmetic apparatus may further comprises a conductive adhesive chip adhered to a surface of each pad, said chip being configured to be in contact with the skin surface. The conductive adhesive chip may comprise a gel layer and a conductive base attached to the gel layer on a side opposed to the pad. In this embodiment, a topical conductive medium such as a gel or cream need not be applied onto the skin surface. Ultrasonic waves can be transmitted to the skin without such a medium because the chip functions as a medium.

The conductive pad may be made of a blast-treated pressed titanium. The pad can be made of any suitable conductive material such as an alumina-based material. However, a titanium-based is preferable because it is unlikely to cause an allergic reaction to the skin. The surface of the pad has preferably fine concavo-convex surface having a more contact surface.

Although it depends on its intended use, it is preferably to provide both of the low-frequency current terminal and the moderate-frequency terminal.

Further, the cosmetic apparatus may further comprise the ultrasonic wave signal generator, although the ultrasonic wave signal generator can be provided separately in a control apparatus. In an embodiment, each of the cosmetic apparatus and the control apparatus can include the ultrasonic wave signal generator, and it is possible to switch these generators.

In another aspect of the present invention, an embodiment of the present invention provides a cosmetic system comprising the cosmetic apparatus defined above and a control apparatus which comprises: the ultrasonic wave signal generator; the low- and/or moderate-frequency current generator; a controller which controls the ultrasonic wave signal generator and the low- and/or moderate-frequency current generator; and an input unit which inputs commands to the controller. In this aspect, the present invention provides a combination of the cosmetic apparatus defined above and a control apparatus. The cosmetic system can further comprise a terminal connected to a ground. When transmitting a low- and/or moderate-frequency current, the body may need to be grounded. Thus, a terminal connected to the ground can be provided separately from the cosmetic apparatus. This may be in the form of a wrist band configured to be attached to the skin surface or a conductive adhesive configured to adhere to the skin surface.

The ultrasonic wave signal generator may transmit two signals corresponding to different frequencies of ultrasonic waves. The frequencies may be within the range of 500 kHz to 10 MHz, preferably 1 MHz to 5 MHz, including about 1.5 MHz, about 2 MHz, and about 3 MHz, and ranges between any two numbers of the foregoing (the different frequencies may be about 3 MHz and about 1.5 MHz). The low-frequency current may have a frequency of about 1 Hz to about 1,000 Hz, including about 50 Hz, about 100 Hz, about 200 Hz, and any ranges between any two numbers of the foregoing. The moderate-frequency current may have a frequency of about 1 kHz to about 10 kHz, including about 5 kHz, and any ranges between any two numbers of the foregoing such as 5 kHz to 10 kHz.

The low- and/or moderate-frequency current generator may be comprised of both a low-frequency current generator and a moderate-frequency current generator. The controller may activate the multiple ultrasonic transducers in sequential cycles. The controller may activate the low- and/or moderate-frequency current generator intermittently in cycles. The controller may activate the ultrasonic transducer to control the intensity of the ultrasonic waves in solenoid curves. Further, the controller may activate the low- and/or moderate-frequency current generator intermittently in cycles, wherein the activation cycles of the ultrasonic transducers and the intermittent cycles of the low- and/or moderate-frequency current generator are synchronized.

In an embodiment, one pad is provided per ultrasonic transducer, and the cosmetic apparatus comprises a plurality of pads arranged on a support, wherein ultrasonic waves and a low- and/or moderate-frequency current are transmitted to each pad in sequential patterns.

The present invention can be applied to a method where a cosmetic method using the cosmetic apparatus defined above, comprises in an embodiment: (i) applying a conductive topical medium on a skin surface; (ii) placing the pad on the medium-applied skin surface; (iii) activating the multiple ultrasonic transducers in sequence to transmit ultrasonic waves in patterns to the medium-applied skin surface through the pad; and (iv) transmitting in patterns a low- and/or moderate-frequency current to the medium-applied skin surface through the pad in combination with the ultrasonic waves while sliding the pad against the medium-applied skin surface. Two signals corresponding to different frequencies of ultrasonic waves may be transmitted to the skin surface. The various embodiments described above can be equally applied to the method.

In another embodiment, the present invention provides a cosmetic method using the cosmetic apparatus defined above wherein multiple pads each provided with one ultrasonic transducer are arranged on a longitudinal support. The method may comprise: (i) applying a conductive topical medium on a skin surface; (ii) placing the longitudinal pad on the medium-applied skin surface; and (iii) transmitting in sequential patterns ultrasonic waves and a low- and/or moderate-frequency current to the medium-applied skin surface through the pads.

In still another embodiment, the present invention provides a cosmetic method using the cosmetic apparatus defined above where a conductive adhesive chip is attached to each pad. The method may comprise: (i) placing the pads with the chips on the skin surface without a conductive topical medium applied on the skin surface; and (ii) transmitting in sequential patterns ultrasonic waves and a low- and/or moderate-frequency current to the medium-applied skin surface through the pads and the chips.

For purposes of summarizing the invention and the advantages achieved over the related art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Further aspects, features and advantages of this invention will become apparent from the detailed description of the preferred embodiments which follow.

### BRIEF DESCRIPTION OF THE DRAWINGS

These and other features of this invention will now be described with reference to the drawings of preferred embodiments which are intended to illustrate and not to limit the invention.

FIG. 1 is a plain view (with a cutaway) of a first embodiment according to the present invention.

FIG. 2 is a lateral view (with a cutaway) of FIG. 1.

FIG. 3 is a bottom view of FIG. 1.

FIG. 4 is an explanatory diagram showing positional relationship between the pad 110 and respective ultrasonic transducers 111, 112, 113 in an embodiment of the present invention.

FIG. 5 is a break apart view showing an embodiment of the cosmetic apparatus (a handset) of FIG. 1, wherein one transducer is shown although three transducers are installed.

FIG. 6A is a wiring diagram showing a main electrical configuration of a cosmetic apparatus according to an embodiment of the present invention. FIG. 6B is a schematic view of a ground separately attached to the body.

FIG. 7 is a time chart showing how feeding of ultrasonic vibration to three ultrasonic transducers 111, 112, 113 is timed and how feeding of low-frequency current or moderate-frequency current to the pad 110 is timed according to an embodiment of the present invention.

FIG. 8 is an explanatory diagram showing a waveform of oscillation strength of the ultrasonic vibration according to an embodiment of the present invention.

FIG. 9 is a longitudinal cross-section of a second embodiment of the cosmetic apparatus according to the present invention.

FIG. 10A is a wiring diagram showing main electrical configuration of the cosmetic apparatus according to an embodiment of the present invention. FIG. 10B is a schematic view of a ground separately attached to the body.

FIG. 11A is a plain view showing a third embodiment of the cosmetic apparatus according to the present invention with a part of the apparatus omitted. FIG. 11B is a schematic view of a ground separately attached to the body.

FIG. 12 is a partial cross-section showing a construction of securing the pads 10 to the belt 11 according to an embodiment of the present invention.

FIG. 13 is a wiring diagram showing main electrical configuration of the cosmetic apparatus according to an embodiment of the present invention.

FIG. 14 is a time chart showing how feeding of ultrasonic vibration to respective pads 10 is timed (i.e., timing in which respective ultrasonic transducers 13 are vibrated) and how feeding of low-frequency current or moderate-frequency current to respective pads 10 successively is timed according to an embodiment of the present invention.

FIG. 15A is a plain view showing a fourth embodiment of the cosmetic apparatus according to the present invention with a part of the apparatus omitted. FIG. 15B is a side view of the cosmetic apparatus of FIG. 15A.

FIG. 16 is a schematic side view of a conductive adhesive chip in an embodiment of the present invention.

Explanation of symbols used is as follows: 1: Main body; 2: Grip; 10: Pad; 11: Belt; 12: Supporting plate; 13: Ultrasonic transducer; 15: Flat fastener; 16: Ring member; 18: Conductive member; 31: Input portion; 32: Ultrasonic signal generating portion; 33: Controller; 34: Oscillator; 35: Ring counter; 36: FET switch; 37: Low-frequency current generating portion; 38: Switch; 39: Switch; 40: Moderate-frequency current generating portion; 101: Main body; 102: Grip; 110: Pad; 111: Ultrasonic transducer; 112: Ultrasonic transducer; 113: Ultrasonic transducer (111, 112, 113 can be used as a low- and/or moderate frequency terminal); 120: Case; 121: Cylindrical member; 122: Conductive member; 123: Conductor; 131: Flat fastener; 132: Grip band; 133: Board; 134: Signal wire; 135: Cord; 140: Ultrasonic signal generating portion; 141: Ultrasonic signal generating portion; 142: Low-frequency current generating portion; 143: Controller; 144: Input portion; 145: FET switch; 146: Ring counter; 147: Oscillator; 149: Moderate-frequency current generating portion; 151: Switch; 152: Switch; 160: First pad; 161: Second pad; 162: First ultrasonic transducer; 163: Second ultrasonic transducer; 171: Gripper.

### DETAILED DESCRIPTION OF THE PREFERRED EMBODIMENT

As explained above, the present invention includes various embodiments, each element of which can be interchangeably used in another embodiment. However, the present invention should not be limited to these embodiments. For example, an embodiment is a cosmetic apparatus, which brings about cosmetic effect by applying ultrasonic vibration to the skin by sliding a pad, to which ultrasonic vibration is fed, against the skin, and which is characterized by possessing a low-frequency current generating portion feeding low-frequency current to the pad.

Another embodiment further possesses a moderate-frequency current generating portion feeding moderate-frequency current to the pad. Ultrasonic waves, a low-frequency current, and a moderate-frequency current have different effects as shown below. By combining these three energies, more significant cosmetic effect can be exhibited. A low-frequency current and a moderate-frequency current may be applied at about 5 V to about 50 V, preferably about 10 V to about 30 V (e.g., about 20 V). Ultrasonic waves may be generated at about 0.05 W to about 5 W, preferably about 0.1 W to about 1 W, depending on its frequency.

| | Low-frequency | Moderate-frequency | |
|---|---|---|---|
| | current | current | Ultrasonic waves |
| Frequency | 1-1,000 Hz | 1-10 kHz | 18 kHz or higher |
| Thermal effect | None | Slightly | Clearly |
| Sensation | Tingling | Tinkling | None |
| Permeability | Surface | Deep layers | Deep layers |

Still another embodiment is a cosmetic apparatus which brings about cosmetic effect by applying ultrasonic vibration to the skin by sliding a pad, to which ultrasonic vibration is fed, against the skin, and which is characterized by possessing a gripper, a pad provided in the gripper, an ultrasonic transducer provided inside the pad, an ultrasonic signal generating portion feeding ultrasonic signals to the ultrasonic transducer, and a low-frequency generating portion feeding a low-frequency current to the pad.

Yet another embodiment further possesses a moderate-frequency current generating portion feeding moderate-frequency current to the pad.

In additional embodiment, multiple ultrasonic transducers are provided inside the pad, and the ultrasonic signal generating portion feeds ultrasonic signals to multiple ultrasonic transducers successively.

Another embodiment is a cosmetic apparatus which brings about cosmetic effect by applying ultrasonic vibration to the skin by sliding pads, to which ultrasonic vibration is fed, against the skin, and which is characterized by possessing a gripper, a first pad provided at one edge of the top of the gripper, the first ultrasonic transducer provided inside the first pad, the first ultrasonic signal generating portion feeding ultrasonic signals at the first frequency to the first ultrasonic transducer, a second pad provided in a position facing the first pad in the top of the gripper, the second ultrasonic transducer provided inside the second pad, the second ultrasonic signal generating portion feeding ultrasonic signals at the second frequency to the second ultrasonic transducer, and a low-frequency current generating portion feeding low-frequency current to the first pad or the second pad.

A further embodiment further possesses a moderate-frequency current generating portion feeding moderate-frequency current to the first pad or the second pad.

Still another embodiment is characterized by possessing a belt-shaped supporting member which can be put on the body, multiple pads locked in the supporting member, multiple ultrasonic transducers respectively provided inside the multiple pads, an ultrasonic signal generating portion feeding ultrasonic signals to the multiple ultrasonic transducers successively, and a low-frequency current generating portion feeding low-frequency current to the multiple pads successively.

Yet another embodiment further possesses a moderate-frequency current generating portion feeding moderate-frequency current to the multiple pads successively.

Further, an embodiment provides a conductive adhesive chip attached to the pad so that a topical conductive medium can be eliminated. This embodiment is preferably when a plurality of pads are used each being provided with one ultrasonic transducer arranged on a longitudinal support.

The present invention is described in detail with reference to the drawings. However, the present invention should not be limited to the drawings. Any elements used in an embodiment can be interchangeably used in another embodiment as long as application of such elements is feasible.

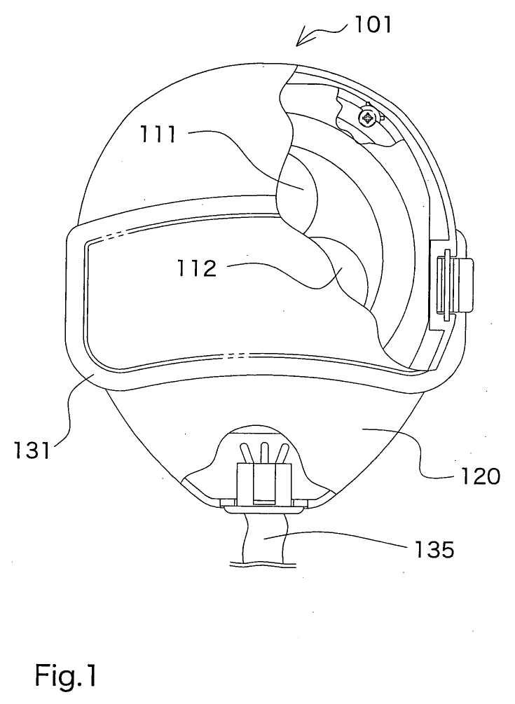

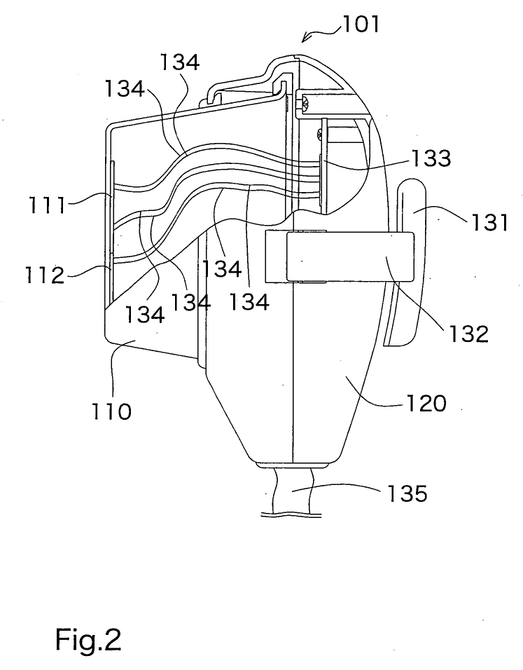

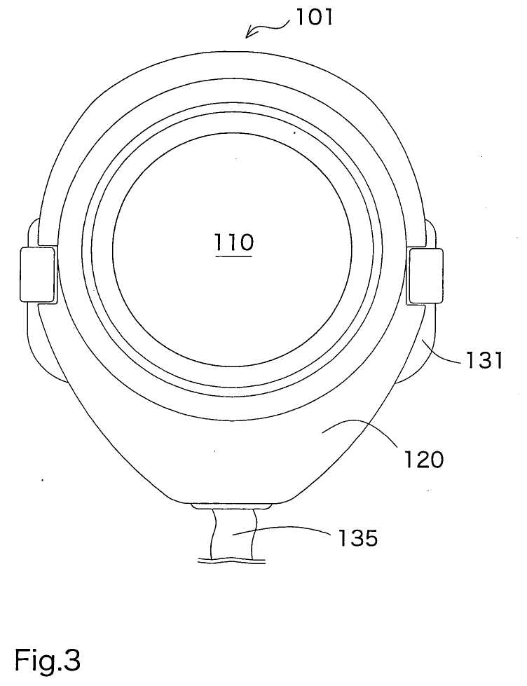

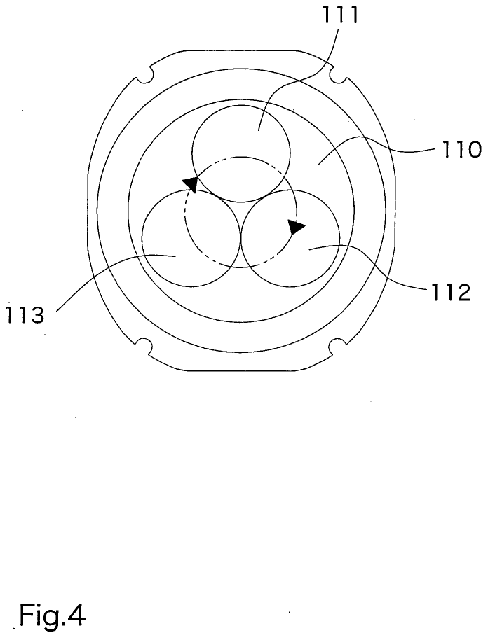

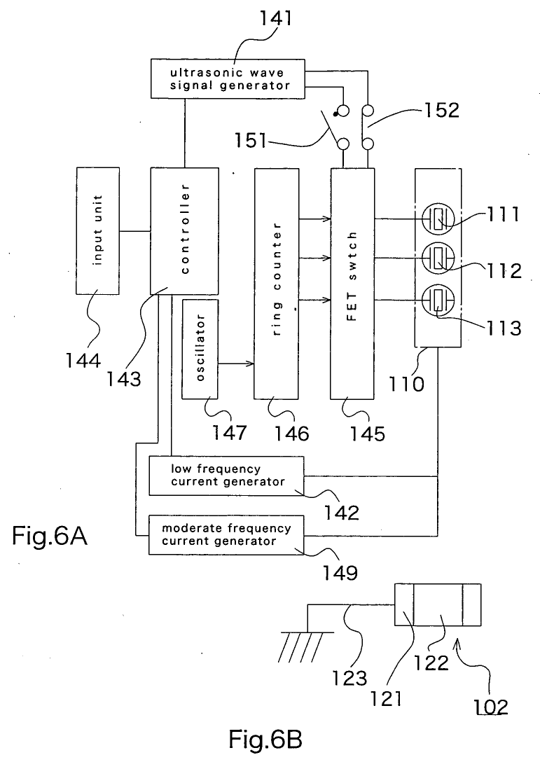

FIG. 1 is a plain view (with a cutaway) of a first embodiment according to the present invention. FIG. 2 is a lateral view (with a cutaway) of FIG. 1. FIG. 3 is a bottom view of FIG. 1. FIG. 4 is an explanatory diagram showing positional relationship between the pad 110 and respective ultrasonic transducers 111, 112, 113 according to an embodiment. FIG. 6 is a wiring diagram showing main electrical configuration of the cosmetic apparatus according to an embodiment.

This cosmetic apparatus brings about cosmetic effect by applying ultrasonic vibration to the skin by sliding a pad, to which ultrasonic vibration is fed, against the skin and comprises a main body 101 (FIG. 6A) and a grip 102 (FIG. 6B). The main body 101 is used for bringing the pad 110 into contact with the skin for the purpose of applying ultrasonic vibration, low-frequency current and moderate-frequency current to the body of a person receiving the cosmetic treatment. The grip 102 functions as a derivation path for low-frequency current or moderate-frequency current when low-frequency current or moderate-frequency current is applied to the body from the pad 110, and possesses an electrically conductive member 122 attached to a peripheral portion of a cylindrical member 121 and connected to a ground via a conductor 123. This cylindrical member 121 coated with the conductive member 122 can be placed around an arm, so that an electric current passes through the body from the pad to the grip. The grip need not be in this shape and can be in any type or shape. In another example, the grip can be built-in in a belt where pads are arranged.

The above-mentioned main body 101 possesses a case 120, the pad 110 provided at one edge of the case 120, three ultrasonic transducers 111, 112, 113 provided inside the pad 110, and an ultrasonic transducer drive mechanism for vibrating these ultrasonic transducers 111, 112, 113 successively.

A grip band 131 is attached to the gripper 120. The case 120 is constructed so that an operator can support it more securely by inserting his/her hand between the case 120 and the grip band 131. The case 120 and the grip band 131 are joined through a flat fastener 132. Consequently, an interval between the case 120 and the grip band 131 can be adjusted to fit in the operator's hand size.

The pad 110 is produced, for example, by applying blast processing to pressed titanium. The pad can be made of any suitable conductive material such as an alumina-based metal-plated material. However, titanium is preferable because it dose not cause an allergic reaction, it can remain clean (by exposing the titanium pad to light, it can be kept clean, and it does not produce a sharp edge even when it is scratched. By blasting treatment using fine glass beads, for example, the surface of titanium can be concavo-convex. Further, the surface can be treated with a mixture of hydrofluoric acid and nitric acid to remove an oxide film and further remove sharp edges, thereby obtaining a relatively smooth surface. Blasting treatment described in paragraphs 10 and 27-31 of JP Laid-open No. 2001-252324 can be used in the present invention. These paragraphs are incorporated herein by reference.

On the inward side of the pad, three ultrasonic transducers 111, 112, 113 are provided at even intervals. These ultrasonic transducers 111, 112, 113 are respectively connected to a board 133 on which an electric circuit, etc. described later are formed via a pair of signal wires 134. Additionally, the board 133 is connected to a power source, etc. through a cord 135. A low- and/or moderate-frequency current passes through the cord 35, the board 133, and wires 134 different from the ultrasonic signal wires 134, and reaches the ultrasonic transducers 111, 112, 113. In an embodiment, the low- and/or moderate-frequency current can be transmitted to the conductive pad via a terminal different from the ultrasonic transducers.

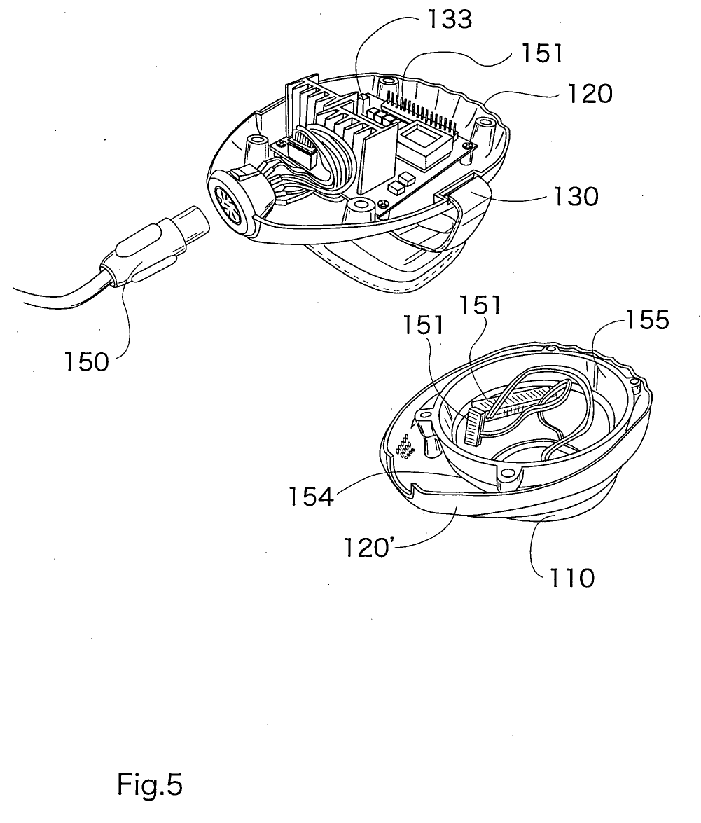

FIG. 5 is a break apart view of an embodiment of the cosmetic apparatus (a handset) of FIG. 1, wherein one ultrasonic transducer is shown although three transducers are installed. In this figure, the handset is configured to be connected to a main control apparatus via a cable (e.g., 8 pins) 150. The handset includes an upper case 120 to which the board 133 is installed, and a lower case 120′ to which a sonic head or conductive pad (made of titanium) 110 is provided which is insulated by a silicon ring 154. Between the upper case 120 and the lower case 120′, a spacer 155 is provided. The upper case 120 has the grip hand 132. The board 133 has terminals for ultrasonic signal waves and a low- and/or moderate-frequency current, which are in the form of harness 151. Male harness is provided on the board 133, whereas female harness is provided at the end of wires which lead to the ultrasonic transducers and the conductive pad 110. Further, the board 133 includes an ultrasonic signal wave generator. The control apparatus may control patterns (e.g., intermittent patterns, cycles, sequences), curves (e.g., sine curves), and intensity of ultrasonic waves, and cycles and intensity of a low- and/or moderate-frequency current. The handset can include an on-off switch.

In addition, in an embodiment, a cooling system can be installed such as a water circular jacket, a blowing fan, etc. so that the surface of the pad can remain cool.

As shown in FIG. 6A, this cosmetic apparatus possesses an ultrasonic signal generating portion 141 generating ultrasonic signals, a low-frequency current generating portion 142 generating low-frequency current, a moderate-frequency current generating portion 149 generating moderate-frequency current, a controller 143 functioning as a controlling portion for controlling these ultrasonic signal generating portion 141, low-frequency current generating portion 142 and moderate-frequency current generating portion 149, and an input portion 144 connected to the controller 143 and used for adjusting a waveform of oscillation strength of ultrasonic vibration, which is described later.

Additionally, this cosmetic apparatus possesses an FET switch 145, a ring counter 146 used for switching the FET switch 145, and an oscillator 147 feeding approximately 2.5 Hz clock signals, which indicate switching timing to the ring counter 146. These FET switch 145, ring counter 146 and oscillator 147 together function as a drive mechanism for the ultrasonic transducers 111, 112, 113.

In an embodiment, the cosmetic apparatus comprises only the pad 110 including the transducers 111, 112, 113, whereas a control apparatus comprises the remaining elements, i.e., the FET switch 145, the ultrasonic wave signal generator 141, the ring counter 146, the oscillator 147, the low-frequency current generator 142, the moderate-frequency current generator 149, the controller 143, and the input unit 144. In another embodiment, the cosmetic apparatus comprises the ultrasonic wave signal generator 141, the FET switch 145, the ring counter 146, the oscillator 147, and the pad including the transducers 11, 112, 113, whereas the control apparatus comprises the low-frequency current generator 142, the moderate-frequency current generator 149, the controller 143, and the input unit 144. In still another embodiment, the control apparatus comprises the controller 143 and the input unit 144, whereas the cosmetic apparatus comprises the remaining elements. Any other suitable combinations can be adopted.

The ultrasonic signal generating portion 141 generates two different types of ultrasonic signals at 3 MHz and 1.5 MHz frequencies. Ultrasonic waves at a frequency of 3 MHz reach the FET switch 145 via a switch 151 and are fed to three ultrasonic transducers 111, 112, 113 from this FET switch 145. Ultrasonic waves at a frequency of 1.5 MHz reach the FET switch 145 via a switch 152 and are fed to three ultrasonic transducers 111, 112, 113 from this FET switch 145 in this embodiment.

The low-frequency generating portion 142 generates low-frequency current at frequencies of approximately 1 to 50 Hz in this embodiment. The moderate-frequency generating portion 149 generates moderate-frequency current at frequencies of approximately 1 to 10 KHz in this embodiment. These low-frequency current and moderate-frequency current are fed to the pad 10.

In the cosmetic apparatus having such electrical configuration, the ultrasonic signal generating portion 141 and respective ultrasonic transducers 111, 112, 113 are connected successively in the timing later-described by the action of the ring counter 146 and the FET switch 145, vibrating respective ultrasonic transducers 111, 112, 113 successively. At this time, when ultrasonic signals at a frequency of 3 MHz are fed, the switch 151 is closed and the switch 152 is opened. When ultrasonic signals at a frequency of 1.5 MHz are fed, the switch 152 is closed and the switch 151 is opened. Additionally, low-frequency current or moderate-frequency current is fed to the pad 110 from the low-frequency current generating portion 137 or the moderate-frequency current generating portion 149.

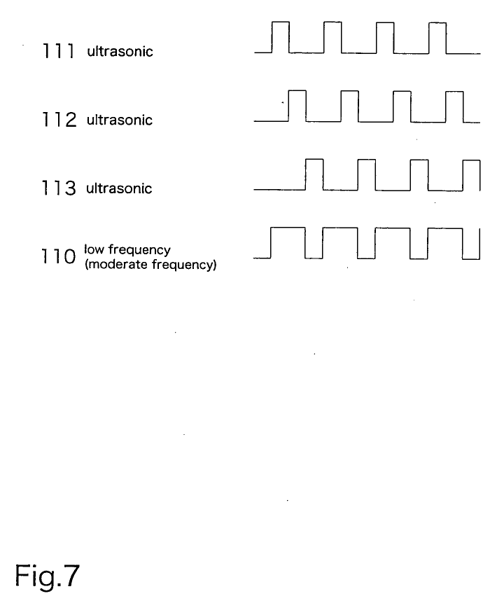

FIG. 7 is a time chart showing how feeding of ultrasonic vibration to three ultrasonic transducers 111, 112, 113 is timed, and how feeding of low-frequency current or moderate-frequency current to the pad 110 is timed, according to an embodiment of the present invention.

As shown in this figure, with three ultrasonic transducers 111, 112, 113 vibrating successively, ultrasonic vibration is fed to different areas in the pad 110 successively. Namely, as indicated by arrows with a chain double-dashed line in FIG. 4, ultrasonic vibration is fed so that the vibration circulates in different areas in the pad 110.

Additionally, low-frequency current or moderate-frequency current is fed to the pad 110 intermittently. Instead of feeding any one of low-frequency current or moderate-frequency current, low-frequency current or moderate-frequency current can be fed alternately, or both of low-frequency current or moderate-frequency current can be fed successively.

Consequently, when sliding the pad 110 against a skin surface of a person who receives the treatment is stopped, no excessive stimulus is given to the skin. Additionally, from a view point of an operator who provides the treatment, ultrasonic vibration is not applied continuously to the same spot of a hand which the cosmetic apparatus is put on and an ultrasonic vibration condition always changes; hence, the operator's skin does not become inflamed even after the operator operates the apparatus for a prolonged period of time. Meanwhile, because any of the three ultrasonic transducers 111, 112, 113 feeds ultrasonic vibration, it becomes possible to give a massage efficiently.

Additionally, because this cosmetic apparatus is constructed to feed low-frequency current or moderate-frequency current to the skin through the pad 110 sliding against the skin, it can feed low-frequency current or moderate-frequency current to a broad skin area, making it possible to achieve slimming effect in a broad body area of the entire body by stimulating the muscle layer of the entire body.

Furthermore, in this cosmetic apparatus, applying low-frequency current or moderate-frequency current to the skin selectively becomes possible. Consequently, it can apply a stimulus to both a surface layer of the muscle layer and a deep layer of the muscle layer, making it possible to achieve a slimming effect to the entire body more effectively.

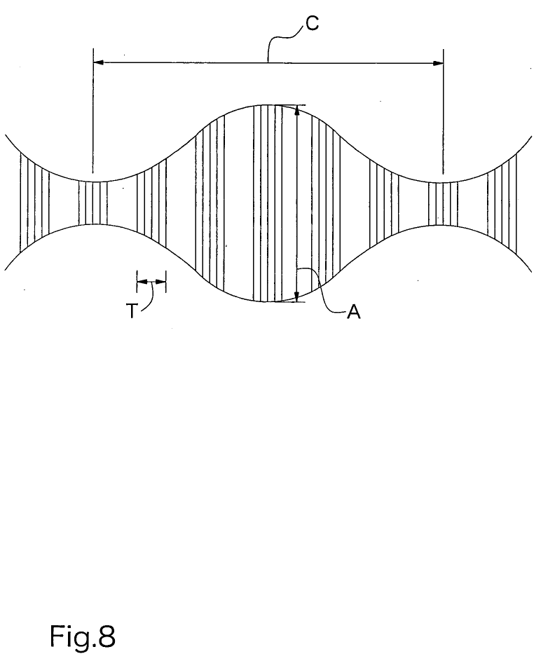

Additionally, intermittent ultrasonic vibration whose oscillation strength changes in a waveform is fed from each pad 110 to the skin. FIG. 8 is an explanatory diagram showing a waveform of oscillation strength of the ultrasonic vibration.

As shown in FIG. 8, signals, whose oscillation strength changes in a waveform and which emit ultrasonic vibration for continuous vibration time T, are fed to each ultrasonic transducer 111, 112 or 113 repeatedly. The waveform of oscillation strength is controlled by the controller 143 shown in FIG. 6.

By inputting a desired value from the input portion 144 connected to the controller 143, Cycle C, Amplitude A and Continuous Vibration Time T for a waveform of the oscillation strength can be adjusted to any values. Consequently, according to a part massaged and physical attributes and others of a person receiving the treatment, giving a massage efficiently becomes possible.

The above-mentioned Continuous Vibration Time T is set, e.g., at several milliseconds to tens milliseconds (1 msec to 100 msec, preferably 5 msec to 50 msec); Vibration Stop Time is also set approximately at the same length of time. A value for the Continuous Vibration Time T is set at a value sufficiently smaller than a value set for the vibration time of each ultrasonic transducer 111, 112, 113 mentioned above. The duty ratio (a ratio of active time period to inactive time period) can be adjusted in the range of 20%-70%, for example.

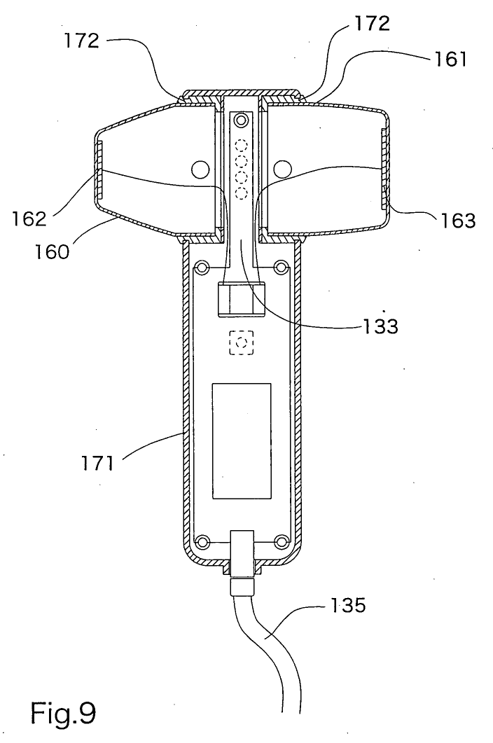

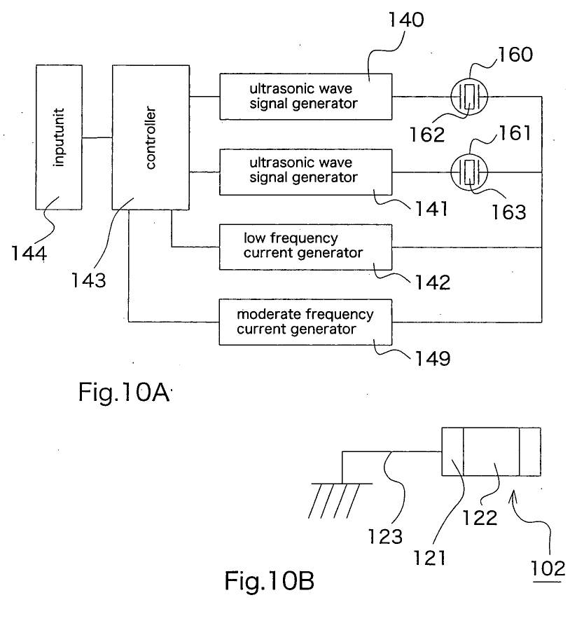

An alternative embodiment of the present invention is described below. FIG. 9 is a longitudinal cross-section of a second embodiment of the cosmetic apparatus according to the present invention. FIG. 10A is a wiring diagram showing main electrical configuration of the cosmetic apparatus. FIG. 10B shows a schematic view of a grip. For the portions identical to those of the first embodiment of the cosmetic apparatus described above, detailed descriptions are omitted by marking them with the same symbols.

The cosmetic apparatus according to the second embodiment possess a gripper 171, the first pad 160 provided at the left edge of the top of the gripper 171, the first ultrasonic transducer 162 provided inside the first pad 160, the second pad 161 provided in a position facing the first pad 160 at the right edge of the top of the gripper 171, and the second ultrasonic transducer 163 provided inside the second pad 161.

Additionally, as shown in FIG. 10B, this cosmetic apparatus possesses a grip 102 of the same sort described above in the first embodiment. The grip 102 functions as a derivation path for low-frequency current or moderate-frequency current when low-frequency current or moderate-frequency current is applied to the body from the pad 110, and possesses an electrically conductive member 122 attached to a peripheral portion of a cylindrical member 121 and connected to a ground via a conductor 123.

The above-mentioned first pad 160 is used for massaging a facial area using its top portion. The first pad 160 is produced in a similar fashion by applying blast processing to pressed titanium.

Additionally, the above-mentioned second pad 161 is used for massaging the body using its top portion. The second pad 161 is produced in a similar fashion used for the first pad 160 by applying blast processing to pressed titanium.

These first pad 160 and second pad 161 are formed to have different shapes so that it makes easier for the user to differentiate the first pad 160 and the second pad 161 when using this ultrasonic cosmetic apparatus.

Additionally, instead of making the first pad 160 and the second pad 161 in different shapes, making the first pad 160 and the second pad 161 in different colors is also acceptable. For example, a smaller pad can be used for face treatment, and a larger pad can be used for body treatment.

The above-mentioned first ultrasonic transducer 162 is attached to the backside of the top portion of the first pad 160. For this first ultrasonic transducer 162, a transducer oscillating ultrasonic vibration at a frequency of 3 MHz, which brings particularly high facial treatment effect to a facial area, is used. For a frequency for this first ultrasonic transducer 162, a frequency at approximately 2 MHz to 4 MHz is preferable for achieving particularly high facial treatment effect to the facial area, according to an embodiment.

The above-mentioned second ultrasonic transducer 163 is attached to the backside of the top portion of the second pad 161. For this second ultrasonic transducer 163, a transducer oscillating ultrasonic vibration at a frequency of 1.5 MHz, which brings particularly high slimming effect to the body, is used. For a frequency for this first ultrasonic transducer 162, a frequency at approximately 0.5 MHz to 2 MHz is preferable for achieving high slimming effect to the body, according to an embodiment.

The above-mentioned first and second pads 160, 161 are connected to the above-mentioned gripper 171 via rubber packing 172. Inside this gripper 171, a controller 143 and others described later are housed.

Additionally, the above-mentioned first and second pads 160, 161 are also used for applying low-frequency current or moderate-frequency current to the body.

As shown in FIG. 10A, this cosmetic apparatus possesses a pair of ultrasonic signal generating portions 140, 141 which generate ultrasonic signals, a low-frequency current generating portion 142 generating low-frequency current, a moderate-frequency current generating portion 149 generating moderate-frequency current, the controller 143 functioning as a control portion for controlling these ultrasonic signal generating portions 140, 141, the low-frequency current generating portion 142 and the moderate-frequency current generating portion 149, and an input portion 144 connected to the controller 143 and used for adjusting a waveform of oscillation strength of ultrasonic vibration.

The low-frequency current generating portion 142 and the moderate-frequency current generating portion 149 can be connected to the transducers 162 and 163 which function as low- and moderate-frequency current terminals. In an embodiment, the low- and moderate-frequency currents can be transmitted directly to the pads 160 and 163 without passing through the ultrasonic transducers.

In an embodiment, the cosmetic apparatus comprises the pads 160 and 163, whereas a control apparatus comprises the remaining elements. In another embodiment, the cosmetic apparatus comprises the ultrasonic signal generating portion 140, the ultrasonic signal generating portion 141, the pad 160 including the transducer 162, and the pad 161 including the transducer 163, whereas the control apparatus comprises the remaining elements. Any other suitable combinations can be adopted.

When massaging a facial area is given using this ultrasonic cosmetic apparatus, 3 MHz ultrasonic vibrations are oscillated from the ultrasonic transducer 162 by operating the first ultrasonic transducer 162. This ultrasonic vibration is propagated to the first pad 160. In this state, the first pad 160 is brought into contact with a facial skin surface to which a gel or a cream is applied in advance. By doing so, the facial skin surface is massaged faster, and high facial treatment effect can be achieved.

When massaging the body is given using this ultrasonic cosmetic apparatus, 1.5 MHz ultrasonic vibrations are oscillated from the ultrasonic transducer 163 by operating the second ultrasonic transducer 163. This ultrasonic vibration is propagated to the second pad 161. In this state, the second pad 161 is brought into contact with a body skin surface to which a gel or a cream is applied in advance. By doing so, the body is massaged at the frequency, which works deeply in the body, and high slimming effect can be achieved.

When a body massage is given, the low-frequency current generating portion 142 generates low-frequency current at a frequency of approximately 1 to 50 Hz, and the moderate-frequency current generating portion 149 generates moderate-frequency current at a frequency of approximately 1 to 10 KHz. These low-frequency current and moderate-frequency current are fed to the second pad 161.

Because this cosmetic apparatus is constructed to feed low-frequency current or moderate-frequency current to the skin through the second pad 161 which is slid against the skin, low-frequency current or moderate-frequency current can be fed to a broad area of the skin, making it possible to achieve slimming effect in a broad area of the entire body.

Furthermore, in this cosmetic apparatus, it becomes possible to apply low-frequency current or moderate-frequency current to the skin selectively. Consequently, a stimulus can be given to both a surface layer of the muscle layer and a deep layer of the muscle layer, making it possible to achieve slimming effect throughout the body more effectively.

Additionally, it can be constructed so that low-frequency current or moderate-frequency current can be fed not only to the second pad 161 but also to the first pad 160.

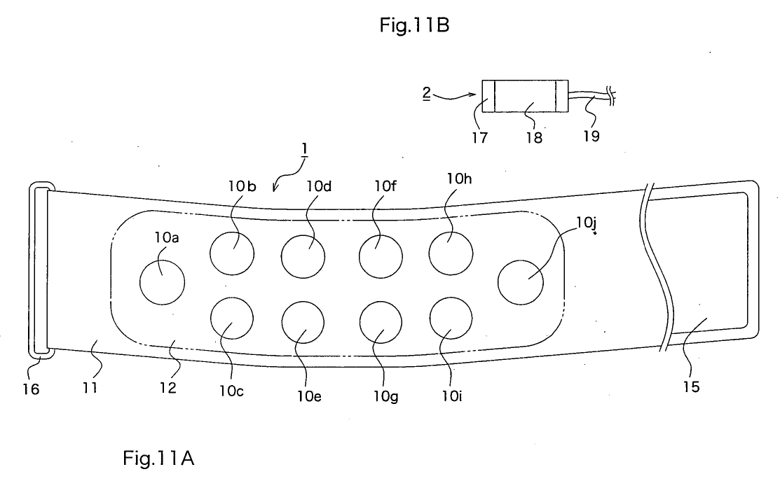

A third embodiment of the present invention is described below. FIG. 11A is a plain view showing the third embodiment of the cosmetic apparatus according to the present invention with a part of the apparatus omitted.

The above-mentioned first and second embodiments are constructed to enable low-frequency current or moderate-frequency current to be fed to a broad area of the entire body by applying low-frequency current or moderate-frequency current to the skin using a pad, which is slid against the skin. In the cosmetic apparatus according to the third embodiment, feeding low-frequency current or moderate-frequency current to multiple pads successively enables low-frequency current or moderate-frequency current to be fed to a broad area in the entire body.

The third embodiment of the cosmetic apparatus according to the present invention comprises a main body 1 and a grip 2. The main body 1 is used for bringing pads 10a, 10b, 10c, 10d, 10e, 10f, 10g, 10h, 10i, 10j into contact with the body for the purpose of applying ultrasonic vibration, low-frequency current and moderate-frequency current to the skin. The grip 2 functions as a derivation path for low-frequency current or moderate-frequency current when low-frequency current or moderate-frequency current is fed to the body from the pads 10a, 10b, 10c, 10d, 10e, 10f, 10g, 10h, 10i, 10j, and possesses an electrically conductive member 18 attached to a peripheral portion of a cylindrical member 121 and connected to a ground via a conductor 19.

The above-mentioned main body 1 possesses a cloth belt 11 and 10 pads 10a, 10b, 10c, 10d, 10e, 10f, 10g, 10h, 10i, 10j (referred to as “pads 10”) attached to the belt 11.

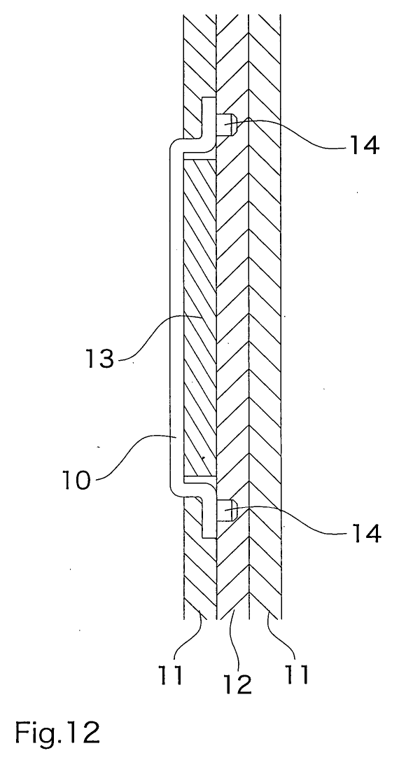

FIG. 12 is a partial cross-section showing a construction for securing the pads 10 to the belt 11.

Each pad 10 is attached onto a supporting plate 12 in a position in which a convex portion 14 provided its back side inserted into a concave portion formed in a surface of the flexible supporting plate 12. This supporting plate 12 is held tightly by the belt 11 comprising a sheet body having right and wrong sides, and is secured. In this position, a surface of each pad 10 is constructed to project exteriorly from a hole portion formed in the belt 11.

This pad is produced, for example, by applying blast processing to pressed titanium.

As shown in FIG. 12, between respective pads 10 and the supporting plate 12, ultrasonic transducers 13a, 13b, 13c, 13d, 13e, 13f, 13g, 13h, 13i, 13j (referred to as “ultrasonic transducers 13”) are provided. Of these ultrasonic transducers 13a, 13b, 13c, 13d, 13e, 13f, 13g, 13h, 13i, 13j, ultrasonic transducers 13a, 13c, 13e, 13g, 13i are used for oscillating ultrasonic waves at a frequency of 2 MHz in an embodiment. Of these ultrasonic transducers 13a, 13b, 13c, 13d, 13e, 13f, 13g, 13h, 13i, 13j, ultrasonic transducers 13b, 13d, 13f, 13h, 13j are used for oscillating ultrasonic waves at a frequency of 1.5 MHz in an embodiment.

With reference to FIG. 11 again, a ring member 16 is provided at one end of the belt 11. A flat fastener 15 is provided at the other end of the belt 11. This flat fastener 15 is designed to lock in a surface of the belt 11. Consequently, after the main body 1 is attached facing the body, by wrapping an end portion on the flat fastener 15 side in the belt 11 into the ring member 16 as well as by locking the flat fastener 15 in a surface of the belt 11, attaching the main body 1 onto the body becomes possible.

FIG. 11B is a schematic view showing a grip 2. A cylindrical member 17 laminated with a conductive material 18 is connected to a ground via a wire 19. This grip 2 can be placed around an arm. However, the grip can be installed in the belt 11 so that a conductive member 18 is in contact with the skin. In this case, the grip is not ring-shaped, but may be shaped in a plate or sheet attached to the belt 11.

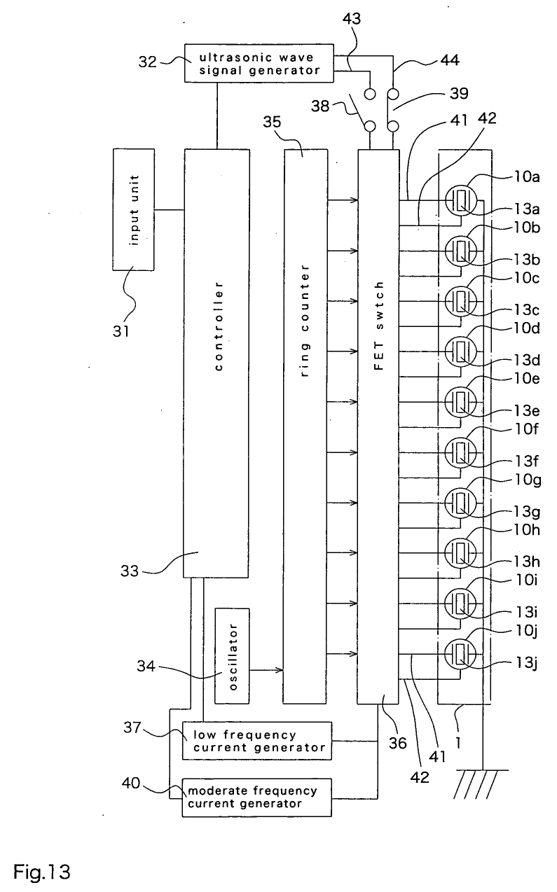

FIG. 13 is a wiring diagram showing main electrical configuration of the cosmetic apparatus.

This cosmetic apparatus possesses an ultrasonic signal generating portion 32 generating ultrasonic signals, a low-frequency current generating portion 37 generating low-frequency current, a moderate-frequency current generating portion 40 generating moderate-frequency current, a controller 33 functioning as a controlling portion for controlling these ultrasonic signal generating portion 32, the low-frequency current generating portion 37 and the moderate-frequency current generating portion 40, and an input portion 31 connected to the controller 33, which is used for adjusting a waveform of the oscillation strength of ultrasonic vibration.

Additionally, this cosmetic apparatus possesses a FET switch 36, a ring counter 35 used for switching this FET switch 36, and an oscillator 34 feeding approximately 2.5 Hz clock signals, which indicate switching timing to the ring counter 35.

In an embodiment, the ultrasonic signal generating portion 32 generates two different types of ultrasonic signals at frequencies of 2 MHz and 1.5 MHz. Ultrasonic waves at a frequency of 2 MHz reach the FET switch 36 via wiring 43 and a switch 38 and are fed to five ultrasonic transducers 13a, 13c, 13e, 13g, 13i from this FET switch 36 and via the wiring 41. Ultrasonic waves at a frequency of 1.5 MHz reach the FET switch 36 via wiring 44 and a switch 39 and are fed to five ultrasonic transducers 13b, 13d, 13f, 13h, 13j from this FET switch 36 and via the wiring 41.

Further, in an embodiment, the low-frequency generating portion 142 generates low-frequency current at frequencies of approximately 1 to 50 Hz. The moderate-frequency generating portion 149 generates moderate-frequency current at frequencies of approximately 1 to 10 KHz. These low-frequency current and moderate-frequency current are fed to the ten pads 10a, 10b, 10c, 10d, 10e, 10f, 10g, 10h, 10i, 10j from the FET switch 36 and via the wiring 42.

In the cosmetic apparatus having such electrical configuration, the ultrasonic signal generating portion 32 and respective ultrasonic transducers 13 are connected successively in the timing later-described by the action of the ring counter 35 and the FET switch 36, and respective ultrasonic transducers 13 vibrate successively. At this time, when the ultrasonic signal generating portion 32 and ultrasonic transducers 13a, 13c, 13e, 13g, 13i are connected, the switch 38 is closed and the switch 39 is opened. When the ultrasonic signal generating portion 32 and ultrasonic transducers 13b, 13d, 13f, 13h, 13j are connected, the switch 39 is closed and the switch 38 is opened.

Additionally, the low-frequency current generating portion 37 or the moderate-frequency current generating portion 40 and respective pads 10 are connected successively in the timing later-described by the action of the ring counter 35 and the FET switch 36; low-frequency current or moderate-frequency current is fed to respective pads 10 successively.

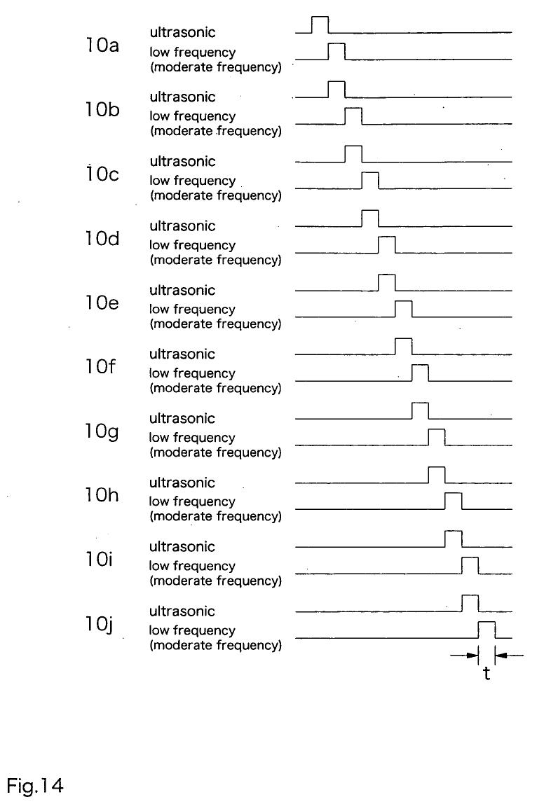

FIG. 14 is a time chart showing how feeding of ultrasonic vibration to respective pads 10 is timed (i.e. timing in which respective ultrasonic transducers 13 are vibrated) and how feeding of low-frequency current or moderate-frequency current to respective pads 10 successively is timed.

As shown in this figure, with respective ultrasonic transducers 13 vibrating successively, ultrasonic vibration is fed to the pads 10 successively. Additionally, low-frequency current or moderate-frequency current is fed to respective pads 10 successively.

In other words, first, ultrasonic vibration is fed to the pad 10a for a period of time T. Time T is 0.4 seconds when a frequency of a clock signal fed to the ring counter 35 from the oscillator 34 is 2.5 Hz. Subsequently, ultrasonic vibration is fed to the pad 10b for a period of time T and low-frequency current or moderate-frequency current is fed to the pad 10a for a period of time T; ultrasonic vibration is fed to the pad 10c for a period of time T and low-frequency current or moderate-frequency current is fed to the pad 10b for a period of time T. By repeating such actions, ultrasonic vibration is fed to ten pads 10a, 10b, 10c, 10d, 10e, 10f, 10g, 10h, 10i, 10j in this order, and low-frequency current or moderate-frequency current is fed to ten pads 10a, 10b, 10c, 10d, 10e, 10f, 10g, 10h, 10i, 10j in this order.

Consequently, although even each pad 10 is designed to be always in contact with the skin, no excessive stimulus is given to the skin. Because any one of ten pads applies ultrasonic vibration or low frequency to the skin all the time, massaging the skin efficiently becomes possible.

Additionally, in a similar manner to the first embodiment, intermittent ultrasonic vibration, whose oscillation strength changes in a waveform, as shown in FIG. 8, is to be fed from respective pads 10.

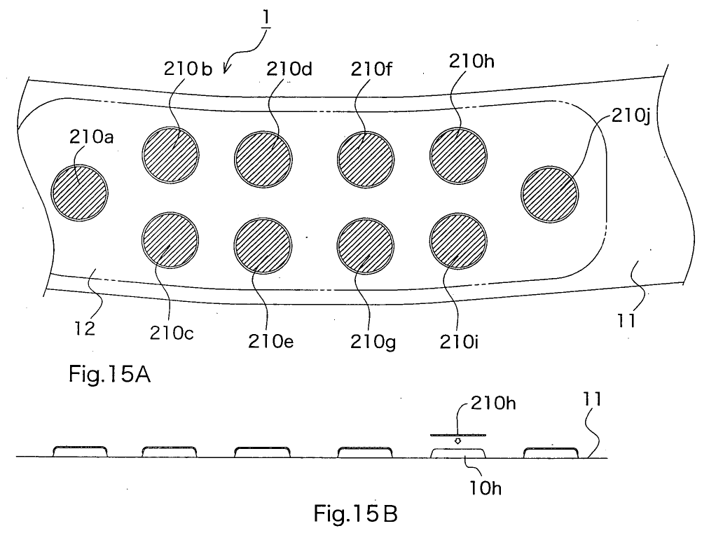

FIG. 15A is a partial schematic view showing another embodiment (a fourth embodiment) of the present invention, wherein a modification is made to the device shown in FIG. 11A. In this figure, conductive adhesive chips 210a, 210b, 210c, 210d, 210e, 210f, 210g, 210h, and 210i are attached to tops of the pads 10a, 10b, 10c, 10d, 10e, 10f, 10g, 10h, and 10i, respectively. These conductive adhesive chips function as energy-transmitting media and eliminate the necessity for use of a gel or cream applied on a surface of a skin. Because the chip is conductive and contains a gel, cream, or other viscous liquid inside the chip, ultrasonic waves and low- and/or moderate-frequency current can be transmitted to the skin layer and muscle layer through the chip without any topical moderate such as a gel, cream, and other applicable liquid applied on the skin. However, in an embodiment, such a topical moderate can be used additionally.

The conductive adhesive chip usable in an embodiment of the present invention may comprise (i) a gel portion made of a conductive gel material such as ion-doped polyurethane and segmented polyether urethane (e.g., alkaline metal salt-containing segmented polyurethane), which may have a thickness of about 0.5 mm to 2.0 mm (preferably about 1.0 mm to about 1.5 mm), and (ii) a reinforcing material such as a nonwoven or woven plastic sheet such as polyester nonwoven sheet, which is embedded in the gel portion, and which may have a weight of about 20 g/cm² to about 100 g/cm², preferably about 30 g/cm² to about 60 g/cm², e.g., 41 g/cm². The reinforcing material need not be used if the gel portion has sufficient mechanical strength. The chip may further comprise a protective film or release film which is laminated on both sides of the gel portion, which is made of a material such as a PET release film which may be transparent and which may have a thickness of about 25 μm to about 250 μm, preferably about 50 μm to about 100 μm, e.g., about 75 μm. The protective film 224 is pealed when attaching the chip to a surface of a skin and a surface of the pad. The used chip can be replaced with a new chip by removing it from the pad. If the protective film is conductive, it need not be removed when in use.

In another embodiment, the pad comprises an energy-transmitting medium inside in place of the chips. The pad can have a compartment storing a gel, cream, or other energy-transmitting moderate which transmits ultrasonic waves and low- and/or moderate-frequency current. Any suitable gel or cream which is usable as a topical moderate for this purpose can be used. In an embodiment, the energy-transmitting moderate is stored and sealed in a thin flat conductive container and an ultrasonic transducer is mounted on top of the container to which low- and/or moderate-frequency current is applied.

In the above described embodiments, the ground is provided separately from the cosmetic apparatus, which is in the form of a wrist band 102, for example. However, the ground can be in any suitable form such as a chip or tape.

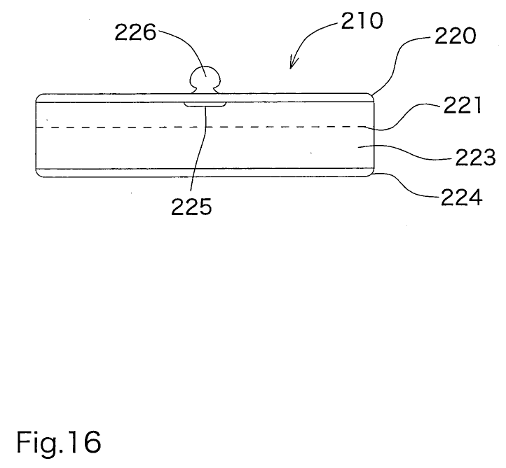

FIG. 16 is a schematic cross sectional view showing a conductive adhesive chip 210 usable in an embodiment of the present invention. This conductive adhesive chip may use the same gel portion and the same reinforcing material described above. In this embodiment, the chip 210 comprises (i) a gel portion 223 made of a conductive gel material such as ion-doped polyurethane and segmented polyether urethane (e.g., alkaline metal salt-containing segmented polyurethane), which may have a thickness of about 0.5 mm to 2.0 mm (preferably about 1.0 mm to about 1.5 mm), (ii) a reinforcing material 221 such as a nonwoven or woven plastic sheet such as polyester nonwoven sheet, which is embedded in the gel portion 223, and which may have a weight of about 20 g/cm² to about 100 g/cm², preferably about 30 g/cm² to about 60 g/cm², e.g., 41 g/cm², (iii) an electrode base 220 made of a material such as a carbon printed PET film, which may have a thickness of about 25 μm to about 250 μm, preferably about 50 μm to about 100 μm, e.g., about 75 μm, which is placed on one side of the gel portion 223, (iv) a conductive tape 225 made of a material such as a conductive polyethylene tape, which is attached to both a surface of the electrode base 220 and a surface of the gel portion 221, and which may have a thickness of about 25 μm to about 250 μm, preferably about 50 μm to about 150 μm, e.g., 100 μm, (v) a conductive projection 226 which is connected to the conductive tape 225 and which is connected to an adapter leading to a ground (this adaptor may be extended from a control apparatus and have a female adaptor press-fitted to the projection), and (vi) a protective film 224 laminated on the other side of the gel portion 223, which is made of a material such as a PET release film which may be transparent and which may have a thickness of about 25 μm to about 250 μm, preferably about 50 μm to about 100 μm, e.g., about 75 μm. The protective film 224 is pealed when attaching the chip to a surface of a skin. The reinforcing material need not be used if the gel portion and the remaining portions have sufficient mechanical strength. The area of the chip can be in the range of about 2 cm² to about 100 cm², preferably about 10 cm² to about 50 cm² (including about 25 cm², i.e., about 5 cm×about 5 cm). The shape of the chip is not limited, and it can be any shape suitable for adhering to a surface of a skin (such as an arm, leg), e.g., a square, a rectangle, or a circle.

As described above, according to an embodiment of the invention, because a low-frequency generating portion feeding low-frequency current to a pad which applies ultrasonic vibration to the skin while sliding against the skin is provided, it becomes possible to achieve slimming effect in a broad area in the body while achieving cosmetic effect.

According to another embodiment of the invention, because a first ultrasonic transducer which oscillates ultrasonic vibration at a first frequency and is provided inside a first pad, a second ultrasonic transducer which oscillates ultrasonic vibration at a second frequency and is provided inside a second pad, and a switching means which switches the first ultrasonic transducer or the second ultrasonic transducer selectively are provided, it becomes possible to provide massage using different frequencies and ultrasonic waves by a single apparatus. Consequently, without increasing apparatus cost and a housing space of the apparatus, or without requiring to switching two different types of ultrasonic cosmetic apparatuses when giving a massage, it becomes possible to achieve high facial treatment effect to the facial area and high slimming effect to the body.

According to still another embodiment of the invention, because a low-frequency current generating portion feeding low-frequency current to a number of pads in order, which applies ultrasonic vibration to the skin successively, is provided, it becomes possible to achieve slimming effect in a broad area of the entire body while achieving cosmetic effect.

According to an additional embodiment of the invention, because a conductive adhesive chip is attached to each of multiple pads, energy can be transmitted to the skin effectively, without applying a conductive topical medium on the skin.

According to yet another embodiment of the invention, because a moderate-frequency current generating portion feeding moderate-frequency current to a pad (or pads) applying ultrasonic vibration to the skin while sliding against the skin is further provided, it becomes possible to achieve slimming effect more effectively by giving a stimulus down to the deep layer portion of the muscle layer.

According to a further embodiment of the invention, because multiple ultrasonic transducers are provided inside a pad, and the above-mentioned ultrasonic signal generating portion feeds ultrasonic signals to the above-mentioned multiple ultrasonic transducers, it becomes possible to achieve cosmetic effect efficiently without giving an excessive stimulus to the skin of a person receiving the treatment and a hand of an operator.

The present application is related to Japanese Patent Application No. 2001-317834, field Oct. 16, 2001, which is published on Apr. 22, 2003 under publication No. 2003-116951. Although the present application does not claim priority to the Japanese application, the disclosure of the Japanese application is incorporated herein by reference in its entirety.

Further, the present application is related to European Patent Application No. 0344726.9, filed Dec. 18, 2003, with regard to an embodiment where a device is in contact with a skin without a gel or cream. The disclosure of the European Patent Application is incorporated herein by reference in its entirety.

It will be understood by those of skill in the art that numerous and various modifications can be made without departing from the spirit of the present invention. Therefore, it should be clearly understood that the forms of the present invention are illustrative only and are not intended to limit the scope of the present invention.

1. A cosmetic apparatus comprising:
at least one conductive pad;
multiple ultrasonic transducers placed on the at least one conductive pad, which is configured to be connected to an ultrasonic wave signal generator to transmit ultrasonic waves to a skin surface through the at least one conductive pad, wherein at least one ultrasonic transducer is placed on each conductive pad; and
a low- and/or moderate-frequency current terminal connected to the at least one conductive pad, which is configured to be connected to a low- and/or moderate-frequency current generator to transmit a low- and/or moderate-frequency current to the skin surface through the at least one conductive pad.

2. The cosmetic apparatus according to claim 1, wherein the at least one conductive pad is one conductive pad, and the multiple ultrasonic transducers are placed on the one conductive pad.

3. The cosmetic apparatus according to claim 1, wherein the conducive pad is made of a blast-treated pressed titanium.

4. The cosmetic apparatus according to claim 2, wherein the multiple ultrasonic transducers are arranged next to each other symmetrically around a center of a surface of the pad facing the skin surface.

5. The cosmetic apparatus according to claim 4, wherein the surface of the pad has a circular shape.

6. The cosmetic apparatus according to claim 1, wherein the at least one conductive pad is multiple conductive pads, each being provided with multiple ultrasonic transducers and a low- and/or moderate-frequency current terminal.

7. The cosmetic apparatus according to claim 6, wherein the multiple pads have different shapes or different colors.

8. The cosmetic apparatus according to claim 2, wherein one pad is provided per ultrasonic transducer, and the cosmetic apparatus comprises a plurality of pads and a support on which the plurality of pads are arranged.

9. The cosmetic apparatus according to claim 8, wherein the support has a longitudinal shape, and the cosmetic apparatus further comprises a belt onto which the support is attached.

10. The cosmetic apparatus according to claim 1, further comprising a gripper configured to be held by a hand.

11. The cosmetic apparatus according to claim 1, wherein both of the low-frequency current terminal and the moderate-frequency terminal are provided.

12. The cosmetic apparatus according to claim 8, further comprising a conductive adhesive chip adhered to a surface of each pad, said chip being configured to be in contact with the skin surface.

13. The cosmetic apparatus according to claim 12, wherein the conductive adhesive chip comprises a gel layer and a conductive base attached to the gel layer on a side opposed to the pad.

14. The cosmetic apparatus according to claim 1, further comprising the ultrasonic wave signal generator.

15. The cosmetic apparatus according to claim 1, wherein the low- and/or moderate-frequency current terminal is provided in the ultrasonic transducer.

16. A cosmetic system comprising the cosmetic apparatus defined in claim 1 and a control apparatus which comprises: the ultrasonic wave signal generator; the low- and/or moderate-frequency current generator; a controller which controls the ultrasonic wave signal generator and the low- and/or moderate-frequency current generator; and an input unit which inputs commands to the controller.

17. The cosmetic system according to claim 16, further comprising a terminal connected to a ground

18. The cosmetic system according to claim 17, wherein the terminal is a conductive adhesive configured to adhere to the skin surface.

19. The cosmetic system according to claim 16, wherein the ultrasonic wave signal generator transmits two signals corresponding to different frequencies of ultrasonic waves.

20. The cosmetic system according to claim 19, wherein the different frequencies are about 3 MHz and about 1.5 MHz.

21. The cosmetic system according to claim 16, wherein the low-frequency current has a frequency of about 1 Hz to about 50 Hz.

22. The cosmetic system according to claim 16, wherein the moderate-frequency current has a frequency of about 1 kHz to about 10 kHz.

23. The cosmetic system according to claim 16, wherein the low- and/or moderate-frequency current generator is comprised of both a low-frequency current generator and a moderate-frequency current generator.

24. The cosmetic system according to claim 16, wherein the controller activates the multiple ultrasonic transducers in sequential cycles.

25. The cosmetic system according to claim 16, wherein the controller activates the low- and/or moderate-frequency current generator intermittently in cycles.

26. The cosmetic system according to claim 16, wherein the controller activates the ultrasonic transducers to control the intensity of the ultrasonic waves in solenoid curves.

27. The cosmetic system according to claim 26, wherein the controller activates the ultrasonic transducers intermittently in cycles.

28. The cosmetic system according to claim 24, wherein the controller activates the low- and/or moderate-frequency current generator intermittently in cycles, wherein the activation cycles of the ultrasonic transducers and the intermittent cycles of the low- and/or moderate-frequency current generator are synchronized.

29. The cosmetic system according to claim 24, wherein one pad is provided per ultrasonic transducer, and the cosmetic apparatus comprises a plurality of pads arranged on a support, wherein ultrasonic waves and a low- and/or moderate-frequency current are transmitted to each pad in sequential patterns.

30. A cosmetic method using the cosmetic apparatus defined in claim 1, comprising:
applying a conductive topical medium on a skin surface;
placing the pad on the medium-applied skin surface;
activating the multiple ultrasonic transducers in sequence to transmit ultrasonic waves in patterns to the medium-applied skin surface through the pad while sliding the pad against the medium-applied skin surface; and
transmitting in patterns a low- and/or moderate-frequency current to the medium-applied skin surface through the pad in combination with the ultrasonic waves while sliding the pad against the medium-applied skin surface.

31. The cosmetic method according to claim 30, wherein two signals corresponding to different frequencies of ultrasonic waves are transmitted to the skin surface.

32. The cosmetic method according to claim 30, wherein the different frequencies are about 3 MHz and about 1.5 MHz.

33. The cosmetic method according to claim 30, wherein the low-frequency current has a frequency of about 1 Hz to about 50 Hz.

34. The cosmetic method according to claim 30, wherein the moderate-frequency current has a frequency of about 1 kHz to about 10 kHz.

35. The cosmetic method according to claim 30, wherein the low- and/or moderate-frequency current is comprised of both a low-frequency current and a moderate-frequency current.

36. The cosmetic method according to claim 30, wherein the multiple ultrasonic transducers transmit ultrasonic waves in sequential cycles.

37. The cosmetic method according to claim 30, wherein the low- and/or moderate-frequency current is transmitted intermittently in cycles.

38. The cosmetic method according to claim 30, wherein the intensity of the ultrasonic waves transmitted from the ultrasonic transducer is controlled in solenoid curves.

39. The cosmetic method according to claim 38, wherein the ultrasonic transducer transmits ultrasonic waves intermittently in cycles.

40. The cosmetic method according to claim 36, wherein the low- and/or moderate-frequency current is transmitted intermittently in cycles, wherein the cycles of the ultrasonic waves and the intermittent cycles of the low- and/or moderate-frequency current are synchronized.

41. A cosmetic method using the cosmetic apparatus defined in claim 8, comprising:
applying a conductive topical medium on a skin surface;
placing the longitudinal pad on the medium-applied skin surface; and
transmitting in sequential patterns ultrasonic waves and a low- and/or moderate-frequency current to the medium-applied skin surface through the pads.

42. A cosmetic method using the cosmetic apparatus defined in claim 11, comprising:
placing the pads with the chips on the skin surface without a conductive topical medium applied on the skin surface; and
transmitting in sequential patterns ultrasonic waves and a low- and/or moderate-frequency current to the medium-applied skin surface through the pads and the chips.
